(19) United States
(12) Patent Application Publication
(10) Pub. No.: US 2012/0071741 A1
(43) Pub. Date: Mar. 22, 2012

(54) SLEEP APNEA MONITORING AND DIAGNOSIS BASED ON PULSE OXIMETERY AND TRACHEAL SOUND SIGNALS

(76) Inventors: Zahra Moussavi, Winnipeg (CA); Azadeh Yadollahi, Winnipeg (CA)

(21) Appl. No.: 12/886,640

(22) Filed: Sep. 21, 2010

(51) Int. Cl. A61B 5/1455; A61B 7/04

(52) U.S. Cl. 600/340; 600/586

(57) ABSTRACT

Detection of apnea/hypopnea events to calculate an apnea/hypopnea index is obtained by analysis of breathing pattern of a patient from breathing and snore sounds and a finger probe recording the SaO2 signal. A detector analyzes microphone signals to detect breath, snore and noise sounds in response to a detected drop in the SaO2 level greater than 2% and to extract and analyze the breathing sounds from a limited time period starting prior to the drop of the SaO2 signal and ending at least at the end of each drop. Separated time periods are divided phases with snore sounds and those with breathing sounds and an estimated breathing volume adjacent to a snore phase is used to estimate the airflow of the snore phase. The relative and absolute energy and duration of the sound periods is used to classify the sound periods into the three groups of breath, snore and noise.

This application relates to a method of sleep apnea monitoring and diagnosis based on pulse oximetry and tracheal sound signals.

This application relates to the subject matter of previous U.S. Pat. No. 7,559,903 issued Jul. 14, 2009 by the present inventors which relates to an apparatus for use to monitor respiratory flow without flow measurement and also detecting apnea/hypopnea events.

This application is also related to a co-pending Application filed on the same day as the above patent Ser. No. 11/692,745 filed Mar. 28, 2007 entitled BREATHING SOUND ANALYSIS FOR ESTIMATION OF AIRFLOW RATE.

### BACKGROUND OF THE INVENTION

Previous U.S. Pat. No. 7,559,903 established an acoustic apnea/hypopnea detection by calculating a feature from the tracheal breath sounds representing variation of the corresponding respiratory flow, and use that to detect apnea/hypopnea events. However, in that patent, we did not give a solution on presentation of respiratory flow in the presence of snoring sounds. A true representation of respiratory flow using only tracheal breath sounds without flow measurement requires careful snore sounds as well as other noises detection as they dominate breath sounds and impair the acoustic flow estimation. The present application is concerned with methods which can overcome these difficulties.

Analysis of breathing sounds from a patient for determination of sleep apnea and/or hypopnea is proposed in a paper entitled “Validation of a New System of Tracheal sound Analysis for the diagnosis of Sleep Apnea-Hypopnea Syndrome” by Nakano et al in “SLEEP” Vol 27 No. 5 published in 2004. This constitutes a research paper postulating that sleep apnea can be detected by breathing sound analysis but providing no practical details for a system which may be used in practise. It is believed that no further work has been published and no commercial machine has arisen from this paper.

U.S. Pat. No. 6,290,654 (Karakasoglu) issued Sep. 18^{th} 2001 discloses an apparatus for analyzing sounds to estimate airflow for the purposes of detecting apnea events. It then uses a pattern recognition circuitry to detect patterns indicative of an upcoming apnea event. In this patent two microphones located close to the patient's face and on patient's trachea are used to record respiratory sounds and ambient noise, respectively. The third sensor records oxygen saturation. Two methods based on adaptive filter were applied to remove the ambient noise from respiratory sounds. Then the signal was band-pass filtered and used for airflow estimation. The estimated airflow signals from two sensors and oxygen saturation data were fed to a wavelet filter to extract respiratory features. Then the extracted features along with the logarithm values of the estimated airflow, signals from two sensors and oxygen saturation sensor were applied to a neural network to find normal and abnormal respiratory patterns. In the next step k-means classifier was used to find apnea and hypopnea events in the abnormal respiratory patterns. In this patent after removing background noise from the signals, the signals are fed to a filter bank which consists of a series of filters in the range of 300-1500 Hz with bandwidth of 100 Hz and then the output of the filter with higher signal to noise ratio is selected for flow estimation. Respiratory sounds data below 300 Hz are crucial for flow estimation during shallow breathing which occurs during sleep. Finally in this patent both acoustical signals and oxygen saturation data are used for apnea detection.

In U.S. Pat. No. 5,797,852 (Karakasoglu) assigned to Local Silence Inc filed 1993 and issued 1998 and now expired is disclosed an apparatus for detecting sleep apnea using a first microphone for detection of breathing sounds and a second microphone for cancelling ambient sounds. This patent apparently lead to release of a machine called “Silent Night” which was approved by FDA in 1997 but apparently is no longer available. In this patent a system comprised of two microphones is proposed for apnea detection. The first microphone is placed near the nose and mouth of the subject to record inhaling and exhaling sounds and the second microphone is positioned in the air near the patient to record ambient noise. The data of the second microphone is used to remove ambient noise from the first signal by means of adaptive filtering. Then the filtered signal is applied to a model for estimating flow and classifying as apnea or normal breathing. The way the patent proposes to record signals it is obvious that the author has never done any experiment with the respiratory sounds. In this patent the main signal is recorded from a place “near” mouth and nose. This is a very vague description of the microphone location and will not record any respiratory sounds especially at low flow rates, which is the rate during sleep usually.

A related U.S. Pat. No. 5,844,996 (Enzmann and Karakasoglu) issued 1998 to Sleep Solutions Inc is directed to reducing snoring sounds by counteracting the sounds with negative sounds. This Assignee has a sleep apnea detection system currently on sale called NovaSom QSG but this uses sensors of a conventional nature and does not attempt to analyze breathing sounds. In this patent a method for removing snoring sounds is proposed. The patent consists of two microphones and a speaker. The first microphone is placed near the noise source to record the noise. The recorded noise is analyzed to generate a signal with opposite amplitude and sign and played by the speaker to neutralize noise in the second position. In order to decrease the error, the second microphone is placed in the second position to get the overall signal and noise and compensate for the noise. This patent is about noise cancellation and specially snoring sound, not apnea detection or screening. The first microphone which provides the primary signal is placed near the head of the subject and not in a place suitable for recording respiratory sounds. Nothing is done for flow estimation or apnea detection.

U.S. Pat. No. 6,241,683 (Macklem) issued Jun. 5^{th} 2001 discloses a method for estimating air flow from breathing sounds where the system determines times when sounds are too low to make an accurate determination and uses an interpolation method to fill in the information in these times. Such an arrangement is of course of no value in detecting apnea or hypopnea since it accepts that the information in these times is inaccurate. In this patent tracheal sound is used for estimation of flow ventilation parameters. Although they mentioned their method can be used to detect several respiratory diseases including sleep apnea, their main focus is not on the sleep apnea detection by acoustical means. They do not mention how they are going to remove ambient noise and snoring sounds from the recordings nor the use of oxygen saturation data for further investigations. Also they have used wired microphone placed over trachea. The other difference is in the signal processing method applied for flow estimation. They are using average power of tracheal sound for flow estimation but it has been shown that average power can not follow flow changes accurately. Also in this study the recorded respiratory sounds are bandpass filtered in the range of 200-1000 Hz to remove heart sounds, which results in low accuracy in estimating flow during shallow breathing.

U.S. Pat. No. 6,666,830 (Lehrman) issued Dec. 23^{rd} 2003 discloses an apparatus for analyzing sounds to detect patterns indicative of an upcoming apnea event. It does not attempt to determine an estimate of air flow to actually locate an apnea event but instead attempts to detect changes in sound caused by changes in airflow patterns through the air passages of the patient. In this patent four microphones are located on a collar around the neck to measure respiratory sounds and a sensor is placed close to nostrils to measure airflow. The airflow signal is used to find breathing pattern and the microphones signals are filtered and analyzed to find the onset of apnea event. In this patent snoring and ambient noise detection has not been discussed. This arrangement does not estimate flow from respiratory sounds so that they cannot calculate respiratory parameters such as respiratory volume based on flow data.

Sleep apnea is a common respiratory disorder during sleep, which is described as a cessation of airflow to the lungs that lasts at least for 10 seconds and is associated with at least 4% drop in the blood's oxygen saturation level (SaO2). The current gold standard method for sleep apnea assessment is full night polysomnography (PSG). However, its high cost, inconvenience for patients and immobility have persuaded researchers to seek simple and portable devices to detect sleep apnea.

There are three types of sleep apnea: Obstructive, central and mixed sleep apnea. The most common one is obstructive sleep apnea (OSA), in which respiratory effort exists but there is no resulting respiratory airflow. Central sleep apnea (CSA) is less common, in which respiratory effort does not exist due to the dysfunction of central drive mechanisms and mixed apnea is a combination of both obstructive and central sleep apnea. The severity of sleep apnea is usually measured by apnea-hypopnea index (AHI) which shows the number of apnea and hypopnea events per hour, although the extent of oxygen desaturation and frequency of arousals or any cardiac arrhythmias that may occur as a result of the sleep apnea/hypopnea events are also indicators of sleep apnea severity. Obstructive sleep apnea is highly prevalent in general population, approaching about 24% of men and 9% of women aged 30 to 60 years old with AHI greater than or equal to 5, while the prevalence of OSA syndrome, defined as AHI greater than or equal to 5 and excessive daytime sleepiness is present in at least 4% of men and 2% of women in the general adult population. The main consequences of sleep apnea are daytime sleepiness, increased risk of cardiovascular and cerebrovascular disease, traffic accidents and impaired quality of life.

Full night polysomnography (PSG) is considered as the gold standard method for sleep apnea diagnosis. However, the high cost of PSG, its time consuming and labour intensive nature and the high prevalence of the disorder have resulted in worldwide long waiting lists of patients delaying their timely access to treatment, while there is increasing evidence in the literature to indicate that untreated OSA is associated with significantly increased morbidity and likely mortality. The above mentioned complications have persuaded researchers to look for portable monitoring devices that can detect sleep apnea with comparable accuracy with the PSG but with smaller number of sensors, and eliminate the need for lengthy in lab monitoring for some patients. There are a variety of portable devices for monitoring sleep apnea. Some use only one signal such as nasal airflow SaO2, respiratory sounds or a combination of 2 to 4 signals.

The main signals used in most of the current portable monitoring devices are either the nasal airflow or SaO2 signals. However, nasal airflow may fail to give an accurate estimate of breathing flow rate due to the misplacement of the sensor during the night or in the cases of mouth-breathing. Use of SaO2 as the only signal for sleep apnea diagnosis is not currently recommended by American Academy of Sleep Medicine (AASM) due to its limited specificity and sensitivity. On the other hand, tracheal respiratory sounds convey important information about the pathology and physiology of the airways; hence, their analysis during sleep can reveal useful information about changes in the behaviour of the upper airway of the patient. Also, tracheal sounds can be used for respiratory flow estimation.

The diagnostic performance of tracheal sound and SaO2 signals for apnea/hypopnea detection has been compared. It is shown that tracheal sound analysis has higher sensitivity than pulse oximetry, while SaO2 signal showed higher specificity.

### SUMMARY OF THE INVENTION

It is one object of the invention to provide an apparatus for monitoring the respiratory flow rate of the patients during the entire night as well as detecting apnea/hypopnea events.

According to a first aspect of the invention there is provided an apparatus for use in analysis of breathing pattern of a patient during sleep for detection of apnea/hypopnea events comprising:

a microphone arranged to be located on the patient for generating signals in response to breathing and snore sounds from the patient;

a finger probe Oximeter to be located on the patient's finger for recording the patient's blood SaO2 signal;

a detector module for receiving and analyzing the SaO2 signals and for receiving and analyzing the microphone signals to extract data relating to the breathing;

wherein the detector module is arranged to analyze the SaO2 signal for detecting the drops in the Oxygen level of the patient;

and wherein the detector module is arranged to analyze the microphone signals to detect breath, snore and noise sounds in response to a detected drop in the SaO2 level.

Preferably the detector module is arranged to extract the drops in the SaO2 signal or greater than a predetermined level and to extract and analyze the breathing sounds from a limited time period starting prior to the drop of the SaO2 signal and ending at least at the end of each drop

Preferably the detector module is arranged to calculate from the analysis of the breathing sounds and SaO2 signal an apnea/hypopnea index.

Preferably the index is calculated from the amplitude of SaO2 and the amount of its drop in the time period.

Preferably the drop is at least of the order of 2%.

Preferably the time period is at least of the order of 10 seconds before the drop.

Preferably the detector module is arranged to extract and separate time periods into groups with snore sounds and groups without snore sounds.

Preferably the detector module is arranged to extract and separate time periods and to divide those periods into groups with snore sounds, groups with breathing sounds and groups with noise.

Preferably a weighted average of the groups and the SaO2 drop and amplitude are used to detect apnea/hypopnea events.

Preferably the detector module is arranged to calculate the relative and absolute energy and duration of the sound segments to classify the sound segments into the three groups of breath, snore and noise.

Preferably the detector module is arranged to calculate the energy, number of zero crossing rate (ZCR) and first formant of the sounds in a plurality of separate windows of data, to classify the sound segments into the groups of breath and snore.

Preferably the detector module is arranged to use the Fisher Linear Discriminant (FLD) method to transform the three features into a new 1-dimential space and then minimize the Bayesian error to classify the sound segments into the groups of breath and snore.

Preferably the detector module is arranged to filter extraneous sounds related to high frequency noises and/or heart sounds and movements. Preferably herein the detector module divides the microphone signals into separate windows and uses the log of the variance (LogVar) of the sound in every window of data

Preferably the detector module is arranged to calculate a flow estimate by the equation from the first few breaths of the patient during the wake time at a self-calibration state to estimate the relative amount of airflow for monitoring the patient's breathing pattern.

Preferably the detector module uses an estimated breathing volume in adjacent phases to a snore phase to correctly estimate the airflow of the snore phase.

Preferably the detector module is arranged to use the estimated airflow to detect periods of apnea and/or hypopnea.

Preferably the detector module includes a display of the relative airflow and the detected apnea/hypopnea episodes and other statistical info for a clinician.

Preferably the display is capable of playing the breathing and classified snoring sounds in any zoomed-in or zoomed-out data window.

Preferably the detector module is arranged to display the extracted information about the frequency and duration of apnea/hypopnea episodes, and their association with the level of oximetry data in a separate window for the clinician.

Preferably the microphone is wireless.

Preferably there is provided additionally a microphone to collect lung sounds from the patient.

Preferably there is provided a third microphone arranged to receive sounds from the patient in the vicinity of the patient so as to be sensitive to snoring and ambient noises and wherein the detector module is arranged to use adaptive filtering to extract the signals relating to the snoring and ambient noises from the signals including the breathing sounds, snoring sounds and noises.

Preferably the microphone is arranged to collect tracheal sounds from the neck.

According to a second aspect of the invention there is provided an apparatus for use in analysis of breathing pattern of a patient during sleep for detection of apnea/hypopnea events comprising:

a microphone arranged to be located on the patient for generating signals in response to breathing and snore sounds from the patient;

a detector module for receiving and analyzing the microphone signals to extract data relating to the breathing;

wherein the detector module is arranged to extract and separate time periods and to divide those periods into groups with snore sounds, groups with breathing sounds and groups with noise;

and wherein the detector module is arranged to calculate the relative and absolute energy and duration of the sound periods to classify the sound periods into the three groups of breath, snore and noise.

According to a third aspect of the invention there is provided an apparatus for use in analysis of breathing pattern of a patient during sleep for detection of apnea/hypopnea events comprising:

a microphone arranged to be located on the patient for generating signals in response to breathing and snore sounds from the patient;

a detector module for receiving and analyzing the microphone signals to extract data relating to the breathing;

wherein the detector module is arranged to extract and separate time periods and to divide those periods into at least groups with snore sounds and groups with breathing sounds;

wherein the detector module uses an estimated breathing volume in adjacent phases to a snore phase to correctly estimate the airflow of the snore phase.

In this application as described in detail hereinafter, there is provided a new method for sleep apnea detection and monitoring, which only requires two data channels: tracheal breathing sounds and the pulse oximetry signal. It includes an automated method that uses the energy of breathing sounds signals to segment the signals into sound and silent segments. Then, the sound segments are classified into breath, snore and noise segments. The SaO2 signal is analyzed automatically to find its rises and drops. Finally, a weighted average of different features extracted from breath segments, snore segments and SaO2 signal are used to detect apnea and hypopnea events. The performance is evaluated on the data of 66 patients recorded simultaneously with their full night PSG study data, and the results are compared with those of the PSG. The results show high correlation between the outcomes of our system and those of the PSG. Also, the method has been found to have sensitivity and specificity values of more than 91% in differentiating simple snorers from OSA patients.

Therefore, it can be concluded that the combination of both signals may result in higher sensitivity and specificity for sleep apnea detection. In this paper we present the results of a new ambulatory device (acoustical sleep apnea diagnosis, ASAD) for detection of sleep apnea using tracheal respiratory sounds and blood's SₐO₂ level. The method is simple, fast, and can analyze 8-hours of data (during the entire night) in less than 15 minutes.

The microphone is arranged to be located on the patient's neck (over suprasternal notch) for detecting breathing sounds;

There is provided a finger probe for SaO2 recording;

The detector module being arranged to analyze the signals to generate an estimate of air flow while extracting extraneous sounds related to snoring and/or heart, to estimate the volume in the respiratory phases adjacent to the snoring phases in order to have a true estimate of respiratory volume in and out, to present the relative estimated respiratory flow to monitor breathing pattern of the patient during the night and to analyze the estimated respiratory flow to detect periods of apnea and/or hypopnea;

There is provided a display of the detected apnea/hypopnea episodes along with the related information for a clinician, a display of the relative respiratory flow for the entire night with zoom in and out options, a display of the recorded respiratory and snore sounds with zoom in and out options with the pathological events highlighted in a red color.

The detector module can connect to an interface for transmission of data to different locations.

The display can include a display of airflow versus time is plotted with apnea and hypopnea episodes marked in.

The display can include oximetry data plotted in association with the estimated airflow.

The display is capable of zoom-in and zoom-out functions in the same window for both airflow and oximetry data simultaneously.

The display is capable of playing the breathing and snoring sounds in any zoomed-in or zoomed-out data window.

The display is capable of playing the breathing and snoring sounds in any zoomed-in or zoomed-out data window.

The display is capable of displaying the extracted information about the frequency and duration of apnea/hypopnea episodes, and their association with the level of oximetry data in a separate window for the clinician.

Preferably there is provided additionally a microphone attached to the chest of the patient to collect lung sounds from the patient.

Preferably the transmitter is arranged to compress data for transmission.

Preferably the remote receiver and detector module are arranged to receive signals from a plurality of transmitters at different locations through an organizer module.

Preferably there is provided a third microphone arranged to receive sounds from the patient in the vicinity of the patient so as to be sensitive to snoring and wherein the detector module is arranged to use adaptive filtering to extract the signals relating to the snoring from the signals including both the breathing sounds and the snoring sounds.

Preferably the detector module is arranged to cancel heart sounds.

Preferably the microphone is arranged to be located in the ear of the patient.

Preferably the microphone in the ear includes a transmitter arranged for wireless transmission to a receiver.

The apparatus described hereinafter provides an integrated system to acquire, de-noise, analyze the tracheal respiratory sounds, estimate airflow acoustically, detect apnea episodes, report the duration and frequency of apnea, and to use wireless technology to transfer data to a remote clinical diagnostic center.

In the present invention the main sensor for recording respiratory sounds is located on the trachea or inside the ear which has been found the best location for flow estimation. Also the present sensors are wireless sensors which decrease the movement noises and produce less interference when subject is asleep.

Such a system reduces the need for polysomnography tests, hence reducing the long waiting list for an accurate diagnostic assessment. The apparatus described hereinafter also facilitates studying patients with mobility or behavioural cognitive issues.

Long distance monitoring and diagnostic aid tools provide large financial saving to both the health care system and families. The apparatus described hereinafter provides a novel system to both developing a new and yet simple diagnostic tool for sleep apnea disorder, and also a new way to connect the specialists and physicians with patients either in remote areas or even at their homes. Aside from its obvious benefit for covering the remote areas with equal opportunity for health care, it also reduces the long waiting list for sleep studies. From a public health perspective, non-invasive and inexpensive methods to determine airway responses across all ages and conditions present a major step forward in the management of sleep apnea disorders.

The apparatus described hereinafter provides a portable and wireless medical monitoring device/intelligent diagnostic system that enables clinicians to remotely and accurately diagnose sleep apnea at much less cost and which greatly reduces discomfort and inconvenience to the patient.

The apparatus described hereinafter can pave the way for a new line of research and application that will simplify the measurement techniques to a large degree while enhancing the quality of symptomatic signs of the disease detection and helping an objective diagnosis.

The apparatus described hereinafter provides a novel, integrated diagnostic system to wirelessly acquire, de-noise, analyze tracheal respiratory sounds, estimate airflow acoustically, detect sleep apnea episodes, report the duration and frequency of apnea, and use secure Internet-based networking technologies to transfer data to a remote centralized clinical diagnostic center.

### BRIEF DESCRIPTION OF THE DRAWINGS

FIG. 1 is a schematic of an algorithm for use in a method according to the present invention.

FIG. 2 shows a typical recorded tracheal sounds and the estimated LogVar signal.

FIG. 3 shows the tracheal sound signal along with the segmentation results.

FIG. 4 shows examples of the recorded signals during hypopnea where FIG. 4a shows an SaO2 signal with a drop and rise, (start and end points of the detected drop and rise are marked by triangle and square markers, respectively) FIG. 4b shows the corresponding tracheal sound signal with segmentation vector (red dashed line) and classification results and FIG. 4c shows a spectrogram of the tracheal sound.

FIG. 5 shows the sigmoid functions S₁ and S₂.

FIG. 6 shows the classification accuracy of the method for different values of Thr_{Event} for detecting apnea and hypopnea events.

FIG. 7 shows the scatter plot of the AHI_{ASAD} and AHI_{PSG} values.

FIG. 8 shows Bland-Altman plots between the AHI_{ASAD} and AHI_{PSG}, the solid line shows the average difference and the dashed lines present the mean±1.96 of standard deviation (boundaries of 95% confidence interval) of the difference.

FIG. 9 shows samples of the recorded tracheal sound in a) time and b) time-frequency domains. The sound segments are extracted and marked manually. Insp-Snr, Insp-Br and Exp-Br represent inspiration segments including snore, inspiration and expiration breath segments void of snore, respectively. The dark repeating frequencies in the time-frequency representation of tracheal sounds (b) show the snore sounds' formant frequencies.

### DETAILED DESCRIPTION

### A. Data Acquisition

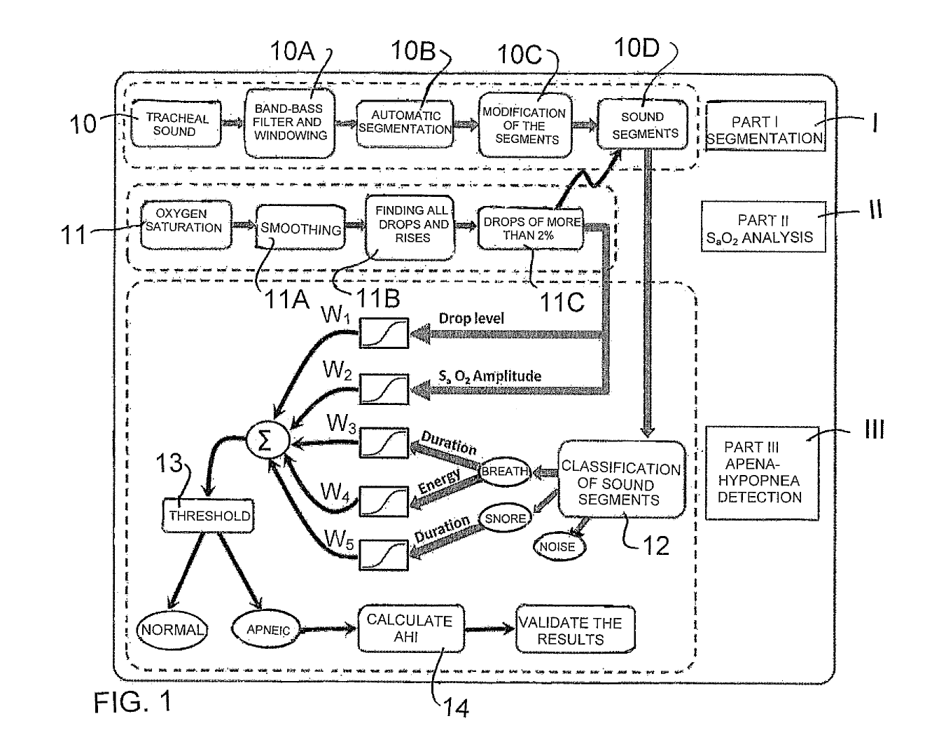

The apparatus of the present invention is shown schematically in FIG. 1 for use in analysis of breathing pattern of a patient during sleep for detection of apnea/hypopnea events. The apparatus includes a microphone 10 arranged to be located on the neck of the patient for generating signals in response to breathing and snore sounds from the patient. The sounds are communicated from the sensor 10 a processor containing software arranged to provide in effect a band pass filter 10A, a system 10B for separating the sounds in to segments, a system 10C for modifying the segments and a transmitter 10D for transmitting the separate segments to a classification system 12.

The apparatus further includes a finger probe Oximeter to be located on the patient's finger for recording the patient's blood SaO2 signal. The signals pass through a smoothing filter 11A and a comparison system 11B, 11C to determine drops in SaO2 signal of more than 2%.

The device further includes a detector module defined by the components 11B, 11C, 10B, 10C, 12 and a threshold detector 13 for receiving and analyzing the SaO2 signals and for receiving and analyzing the microphone signals to extract data relating to the breathing.

As explained in detail hereinafter, the detector module is arranged to analyze the SaO2 signal for detecting the drops in the Oxygen level of the patient and to analyze the microphone signals to detect breath, snore and noise sounds in response to a detected drop in the SaO2 level.

This is carried out using the algorithm described hereinafter and including components I, II and III of FIG. 1.

In particular the detector module is arranged to extract the drops in the SaO2 signal or greater than a predetermined level and to extract and analyze the breathing sounds from a limited time period starting prior to the drop of the SaO2 signal and ending at least at the end of each drop. In particular the detector module is arranged to calculate from the analysis of the breathing sounds and SaO2 signal an apnea/hypopnea index.

### A. Data Recording

Tracheal respiratory sounds are recorded by a small omni-directional microphone (Sony ECM-77B) inserted in a chamber, and attached to the patient's neck over the Suprasternal notch with a double sided adhesive tape. The microphone and chamber are held in place with a soft neckband, which is fastened gently around patient's neck to assure the microphone is not misplaced during the night. The SaO2 signal is recorded with a Masimo finger probe (5N040) connected to a Masimo pulse oximeter (Radical signal extraction pulse oximeter). The sounds are amplified and lowpass filtered with 5 KHz cutoff frequency using Biopac (DA100C) amplifiers. The SaO2 signal and filtered tracheal sounds are simultaneously digitized at a sampling rate of 10240 Hz by National Instruments data acquisition module (NI9217). The digitized signals are saved in a file every 3 minute resulting approximately 140 files for an entire night of recording.

### B. Signal Analysis

The energy of respiratory tracheal sounds in logarithmic scale has been shown to change with respiratory flow rate, and has been used for respiratory flow estimation. Hence, in this study, the logarithm of the tracheal sound variance (LogVar) is used to estimate the relative respiratory flow and the percentage of respiratory flow reduction or complete lack of flow. The procedure of finding apnea-hypopnea events is implemented in three steps:

1) the tracheal sound signal is analyzed to find the sound and silent segments,

2) the SaO2 signal is investigated to find the periods including drops in the blood's oxygen level,

3) the tracheal sound segments corresponding to the periods with reduced SaO2 level are further examined and automatically classified into breath, snore and noise segments; their temporal information along with the features of SaO2 signal are used to determine the apnea and hypopnea events. Details of the method shown in FIG. 1 are discussed in the following sections.

### B.1 Automatic Sound Segmentation

The first step in analyzing respiratory sounds is to remove the effects of low and high-frequency noises. When recording respiratory sounds over the trachea, heart sounds are the main inevitable noises that are picked up by the microphone. Heart sounds are low frequency signals, and overlap with the tracheal sounds in the frequency range below 200 Hz. Different methods have been proposed for heart sounds detection and reduction. However, all of these methods are verified on the data of healthy subjects during wakefulness when subjects are breathing normally, and other noises such as snore, movements or blanket noises are not present. Furthermore, those methods are developed for heart sounds cancellation from lung sound, which is a low frequency signal compared to tracheal sound. Since tracheal sounds have considerable energy components in the frequency range of above 200 Hz and below 1000 Hz the recorded sounds are first bandpass filtered by a Butterworth filter of order 5 and cutoff frequency of 200 to 1000 Hz to reduce the effects of heart sounds and high frequency noises, while including the main frequency components of breath and snore sounds.

The bandpass filtered sound signals are divided into the windows of 20 ms in duration with 75% overlap between the adjacent windows. The values of optimum window size and overlap for segmenting the tracheal sound signal are selected based on the results of our previous studies on acoustical flow estimation. Energy or amplitude of tracheal sounds is usually used to find the sound segments and the breathing cycles. In this study, LogVar is calculated in each window which represents the signal's energy.

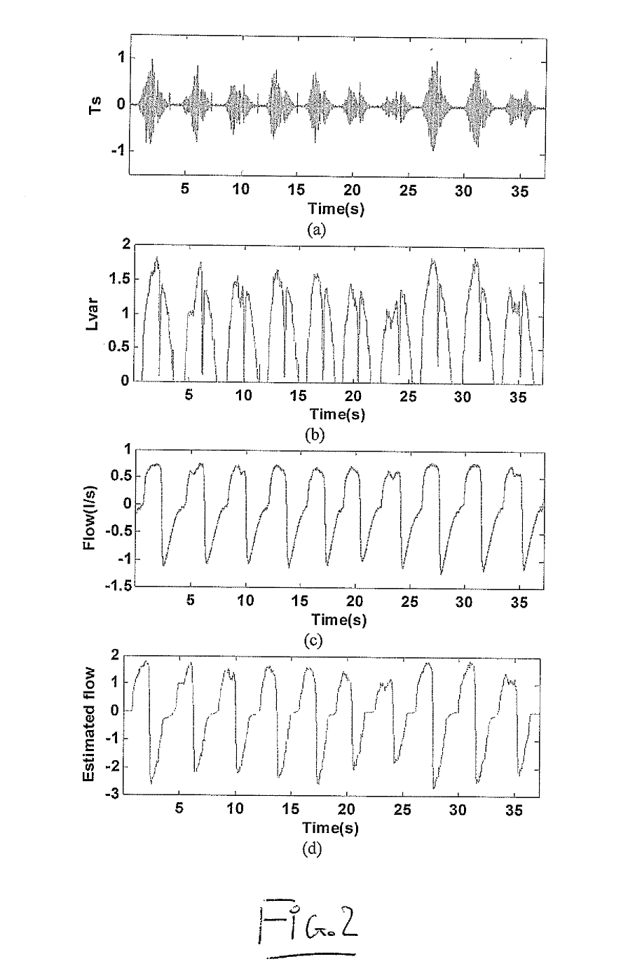

FIG. 2 shows a typical recorded tracheal sounds and the estimated LogVar signal. The data of this particular subject are recorded with the same device during sleep, but including a face mask pneumotachograph connected to a pressure sensor for direct measurement of flow signal for comparison with estimated flow.

Comparing the recorded flow (FIG. 2-c) and the estimated LogVar signals (FIG. 2-b), it can be seen that LogVar follows absolute values of flow signal (FIG. 2-a). FIG. 2-d shows the result of estimating relative flow from the LogVar, which is closely related to the corresponding recorded flow. Note that the amplitude of the estimated flow does not represent the actual amount of flow in L/s as it is the relative flow without calibration.

The median of the LogVar values of all the windows of the bandpass filtered tracheal sound is calculated and used as a threshold to automatically classify each window either as a sound or silent window. Then, if two successive windows classified as sounds are not farther than the length of one window size (20 ms) apart, they are combined together, i.e., the silent portions in the middle are ignored. This process is continued by merging the small segments, i.e., equal to 20 ms, with their adjacent close segments. Since the duration of respiratory phases, i.e., inspiration/expiration, is not usually more than a second, the segments longer than are divided into smaller segments (FIG. 1—Part I).

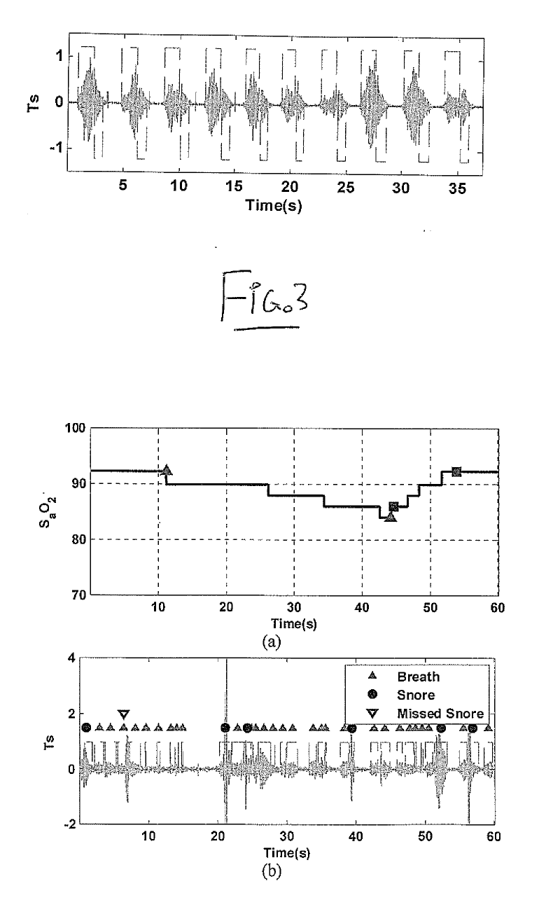

FIG. 3 shows the tracheal sound signal along with the segmentation results. The segmentation vector (dashed line) is multiplied by −1 in successive segments for clarity purposes.

The segmentation performance is verified by manual auditory and visual inspection of the sound signals in the time-frequency domain. The automatic segmentation results are compared with those of the manual detection in terms of absolute delays in detecting the start and end of each sound segment, difference in the duration of each segment and the number of missed segments.

### B.2 Analysis of SaO2 Signal

Both cessation and reduction of airflow should be associated with at least 4% drop in SaO2 signal for being counted as an apnea/hypopnea event. SaO2 signal is a very low frequency signal; hence, to have a fast method it is more efficient to start the data analysis by finding the drops and rises of this signal. The SaO2 signal is smoothed with a median filter in windows of 150 ms. The falling and rising step changes in the SaO2 signal are found automatically by taking the derivative of the signal. The step changes in SaO2 signal which last less than 20 s are merged together to get the start and end point of the fall and rise in the SaO2 signal (FIG. 1—Part II).

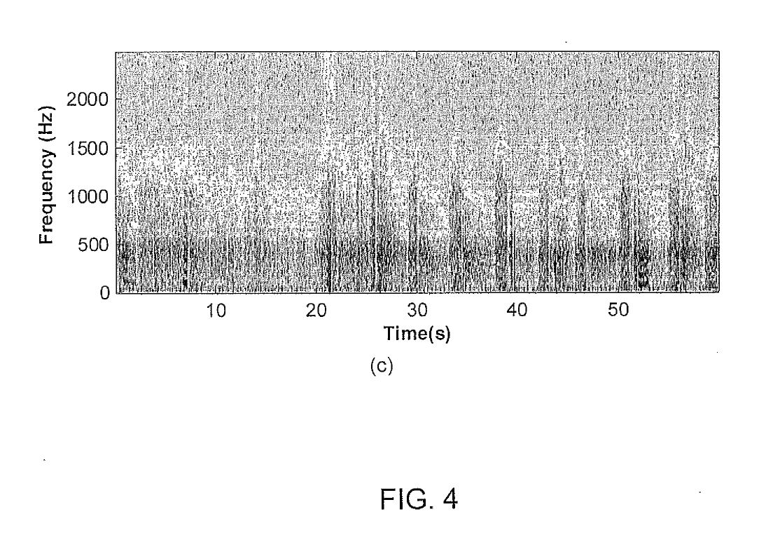

FIG. 4-a shows a period of SaO2 signal which contains a drop and a rise. The detected start and end points of the drop and rise in the SaO2 signal are marked by triangle and square markers, respectively.

The sound signals within the periods of SaO2 drop are subsequently analyzed to examine whether an apnea or hypopnea has occurred in that period or not. To ensure that marginal apnea and hypopnea events are not missed, all the drops of more than 2% are considered. On the other hand, there is usually a delay between the occurrence of apnea or hypopnea event and the drop in SaO2 signal; to consider this the sound segments are examined from 10 s prior to the drop of SaO2 signal (FIG. 1—Part II).

### B.3 Apnea-Hypopnea Detection

### Sound Segments Classification

When recording nocturnal tracheal sounds over the neck, in addition to breath sounds other sounds such as snores and different noises including oral noises, ambient sounds, speech and blanket movements are also captured by the microphone. Oral noises are generally short in duration with large amplitude, movements are long in duration with high amplitudes and speech signals have very large amplitudes compared with the breath sounds. Snore sounds occur in different parts of respiratory cycle and they usually have higher amplitude than breath sounds. When analyzing the sound signals to estimate the respiratory flow, the presence of these additional sounds is a major problem that has to be handled carefully. Therefore, a smart function has been developed to first classify the tracheal sound segments into breath, snore and noise segments. The program uses the sound segments' energy and duration to classify them into different groups of breath, snore and noise.

The amount of change in the tracheal sounds' energy due to the flow variation is different among different people. Therefore, a period of few minutes of breath sounds segments of the subject at the beginning of each recording (when he/she is awake) is used to derive the energy level and the duration of normal breathing of the subject as a reference (self-calibration stage). To classify the sound segments, the segments which are close to each other and could be considered as pairs of successive breathing cycles, are marked. Energies of the pair segments are compared with each other. If the ratio is larger than the average plus standard deviation of the normal breath sound segments' energies (extracted in the self-calibration stage), the segment with greater energy is marked as snore and the other segment is labelled as breath. On the other hand, if the energies of the pair segments are similar and each segment's energy is in the range of normal breath sound segments' energy, they are marked as breath segments. Then, the single segments (with no segment close to them to form a pair) are marked as either snore or breath based on their energies and durations. Finally, the remaining single segments, which are shorter or longer than normal breath sounds, are labelled as noise representing oral or movement noises (FIG. 1—Part III).

FIG. 4-b shows an example of the tracheal sound recorded during hypopnea (corresponding to the SaO2 signal of FIG. 4-a) along with the sound segments classification results. The classification algorithm missed one snore segment and labelled it as breath segment (red downward triangle). Spectrogram of the recorded sounds is presented in FIG. 4-c showing the temporal and spectral changes in the energy components of the breath and snore segments. Also note the time span of 15 to 20 secs, where there is a silent period with no detectable breathing.

### Finding Apnea/Hypopnea Events

Since neither respiratory flow nor snore sounds are present during apnea events, these episodes can be easily detected by finding the periods, in which the sounds energy is below 10% of the reference value (extracted during self-calibration stage) and its duration is more than 10 s. However, detecting hypopnea events is more complicated; they can be either very shallow breathing episodes for more than 10 secs, short durations of normal breathings with periods of no-breathing in between or a combination of mildly shallow breathing and snoring that indicates partial airway obstruction (FIG. 4). All of these conditions may result in a deficiency in breathing and a drop in the blood's SaO2. However, these conditions are different for each person, and depending on position, sleep stage, etc. they can change during the night for the same person.

The following features of the sound segments and SaO2 signal are investigated to distinguish different situations that correspond to apnea/hypopnea event:
The total energy of the breath sound segments (Eng_{Br}) in each period
The duration percentage of the breath sound segments in each period (Dur_{Br})
The duration percentage of the snore sound segments during each period (Dur_{Snr})
The amount of drop in SaO2 signal (Drp_{Sat})
The amplitude of SaO2 signal (Amp_{Sat})

Each feature is transformed with either the sigmoid functions S₁(t) or S₂(t) to represent the importance and contribution of each feature in the occurrence of an event:

$$\begin{matrix}{S}_{1} ( t ) = {( 1 - {e}^{\frac{t - a}{b - a} \times ( {x}_{2} - {x}_{1} ) + {x}_{1}} )}^{- 1} & ( 1 ) \\ {S}_{2} ( t ) = 1 - {S}_{1} ( t ) & ( 2 )\end{matrix}$$

where a and b show the variation range of each parameter and x₁=−10 and x₂=10 are two constants for which the function (1−e^{-t})⁻¹ converges to 0 and 1, respectively. FIG. 5 shows the sigmoid functions S₁ and S₂. The transformed values are weighted and added together:

$$y = {\sum }_{i = 1}^{5} {w}_{i} S ( {f}_{i} ) , \qquad ( 3 )$$

where fᵢ represents different features (Eng_{Br}, Dur_{Br}, Dur_{Snr}, Drp_{Sat} and Amp_{Sat}), S(ƒ₂) is the sigmoid function (S₁ or S₂) and wᵢ is the weighting value of each feature.

The limits of each feature (a,b), wᵢ and choice of sigmoid function are determined heuristically and based on the preliminary information regarding the importance of different features and their association in the occurrence of apnea or hypopnea events. The value of y is compared with a threshold of Thr_{Event}; if it is less than the threshold, the period with the drop in SaO2 signal is considered as normal; otherwise, it is counted as an apnea/hypopnea event (FIG. 1—Part III). To find the Thr_{Event}, different values in the range of [0.2-0.9] are used as thresholds for finding apnea/hypopnea events and calculating the number of events per hour (AHI_{ASAD}). The AHI_{ASAD} values are used to classify the subjects into simple snorers and OSA patients, and the results are compared with the classification results based on the AHI values of the PSG study (AHI_{PSG}) that is manually calculated by the sleep lab technicians. The value of threshold, for which the highest accuracy is achieved, is selected as the Thr_{Event}.

The calculated Thr_{Event} is applied to re-estimate the subjects' apnea and hypopnea events. The AHI_{ASAD} and AHI_{PSG} values are compared in terms of linear correlation and Bland-Altman statistical measure. Bland-Altman measure is designed to measure the agreement between two methods that investigate the same property, and it has been widely used in sleep apnea studies to validate the performance of portable monitoring devices.

One of the main applications of sleep apnea portable monitors is to screen the patients and separate OSA patients from simple snorers for advanced diagnosis. In the last evaluation, the performance of the estimated AHI_{ASAD} values in classifying the subjects into two groups of simple snorers and OSA patients is investigated. Since PSG is considered as the gold standard, the subjects are usually grouped into simple snorers and OSA patients depending on their AHI_{PSG} However, there is no standard threshold of AHI_{PSG} for such grouping. Researchers have used different values of AHI between 5 to 20 as the threshold between simple snorers and OSA patients. Hence, in this study we investigated grouping of the patients with the AHI_{PSG} values of 5, 10, 15 and 20 as the threshold, and determined what AHI_{ASAD} would correspond to those of PSG with the highest accuracy.

For each of the above mentioned four AHI_{PSG} thresholds, data is divided into train and test data sets to find the best value of AHI_{ASAD} corresponding to the selected AHI_{PSG} threshold that gives the best classification of subjects. 6-fold algorithm is used to divide the subjects into train and test data sets. The patients are randomly clustered into 6 groups (11 patients in each group); data of 5 groups are selected as the train data set and the sixth group is considered as the test data set. The training data is used to find the corresponding threshold of AHI_{ASAD} which is applied to classify the subjects in the test data set and find the classification sensitivity and specificity. The receiver operating curve (ROC) and the area under the curve (AUC) are also calculated to evaluate the classifier's performance. This process is repeated for all 6 folds as test data set and the sensitivity, specificity and AUC results are averaged. Finally, to remove the classifier's bias to the choice of train and test data sets, the whole process is repeated 200 times and the results are averaged among all trials.

In the first step of the processing, the recorded signals are segmented into sound and silent segments. A thresholding based technique is used to have a fast algorithm for detecting windows of sound (with the fixed length of 20 ms); this is followed by a smart post-processing to merge the windows and determine continuous segments of sounds with variable lengths that corresponded to different cases such as breath, noise or snore. Table II shows the mean and standard deviation values of the delays, duration errors and missed segments for 3059 breath and 1557 snore segments of 16 subjects. The errors are averaged for all the segments of every subject and among different subjects. The results indicate the method detects more than 96% of the sound segments of different lengths and intensities correctly. Considering that data is recorded in real condition with no control on the position and sleep situation of the subjects or the ambient noise, the results are promising and reliable in detecting the sound segments with a high accuracy. Moreover, the segmentation algorithm is fully automatic and fast which are important factors in studying the overnight data of the patients.

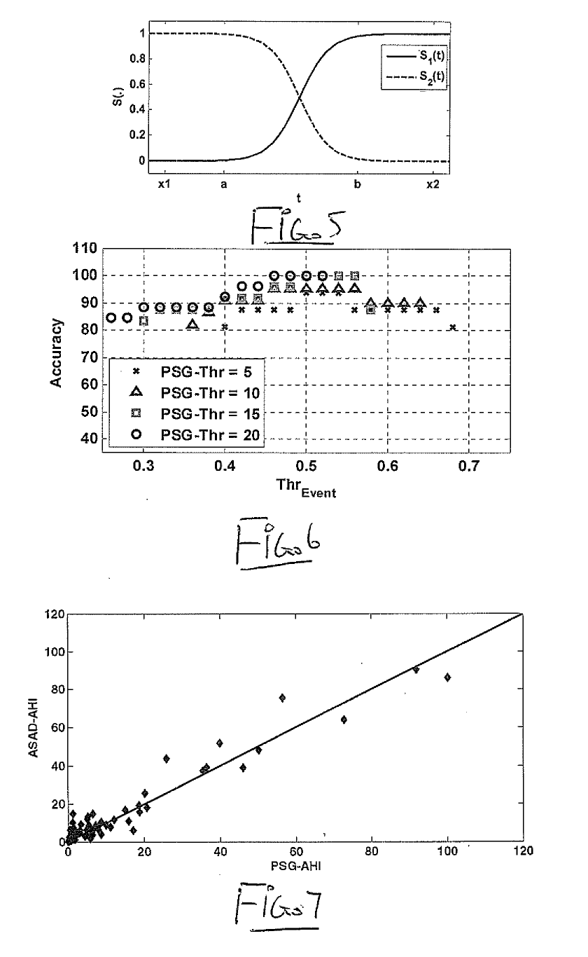

FIG. 6 shows the classification accuracy of the method for different values of Thr_{Event} for detecting apnea and hypopnea events. The classification is performed for different values of AHI_{PSG} and it can be seen that with the threshold of 0.5, the best possible performance is achieved for different cases. This threshold is used in the rest of study for finding apnea/hypopnea events and calculating AHI_{ASAD} values.

TABLE II
Mean and standard deviation values of the automatic segmentation errors.

| | | | | Missed |
|---|---|---|---|---|
| Segment | Start(s) | End(s) | Duration(s) | (%) |
| Breath | 0.250 ± 0.216 | 0.216 ± 0.198 | 0.275 ± 0.178 | 3.42 |
| Snore | 0.253 ± 0.307 | 0.305 ± 0.214 | 0.305 ± 0.214 | 3.10 |

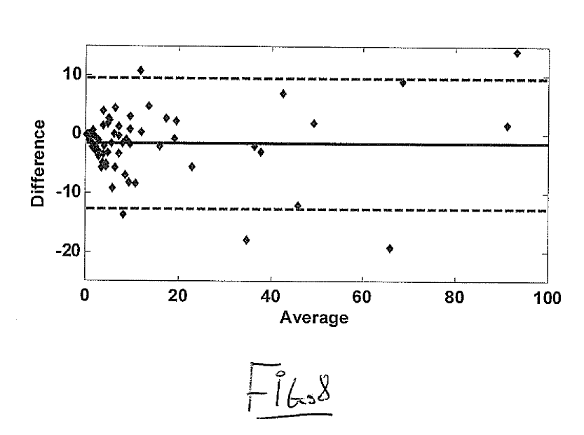

The classified sound segments and SₐO₂ signal are used to determine the occurrence of an apnea or hypopnea event and estimate the AHI value of each subject. The AHI values of the method (AHI_{ASAD}) are compared with those of the PSG study (AHI_{PSG}). FIG. 7 shows the scatter plot of the AHI_{ASAD} and AHI_{PSG} values. The correlation ratio between the AHI_{ASAD} and AHI_{PSG} values is found to be 0.96 (p<0.0001).

Bland-Altman statistical test is performed to verify the agreement between the results of ASAD and PSG systems. The average and standard deviation values are −1.56 and 5.54, respectively, and only 5 out of 66 subjects are outside the 95% confidence interval as expected statistically (FIG. 8). These results confirm high correlation between the AHI_{ASAD} and AHI_{PSG} values.

Finally, AHI_{ASAD} values are used as a threshold to classify the subjects into simple snores and OSA patients. Again the AHI values of the PSG system are used as the gold standard to determine the true classes of the patients. The classification performance of the method is evaluated based on specificity and sensitivity values for four different thresholds of AHI_{PSG} values (5, 10, 15, 20) representing different severity levels of sleep apnea (Table III). The results of Table III show that for the AHI_{PSG} thresholds of more than 10 the AUC is close to 1; this indicates the classifier has high sensitivity and specificity. For the AHI_{PSG} thresholds of more than 20 the sensitivity and specificity of the classifiers are found to be more than 91%. The high sensitivity and specificity of the classifier is expected as the AHI values calculated are highly correlated with those of the PSG system.

TABLE III
Average ± standard deviation of specificity and sensitivity values of
ASAD system for different thresholds of AHI_{PSG} and AHI_{ASAD}.
The classification is repeated 200 times and the results are averaged.

| | AHI_{PSG} | | | |
|---|---|---|---|---|
| | 5 | 10 | 15 | 20 |
| | AHI_{ASAD} | | | |
| | 8.6 | 13.0 | 18.5 | 23.0 |
| Sensitivity | 74.3 ± 2.7 | 82.8 ± 6.5 | 84.6 ± 7.5 | 91.6 ± 10.7 |
| Specificity | 82.4 ± 5.3 | 91.1 ± 1.9 | 96.0 ± 2.8 | 97.8 ± 0.8 |
| AUC | 0.87 | 0.95 | 0.96 | 0.99 |

A new automatic acoustic method is provided to detect apnea and hypopnea events with no need for respiratory flow measurement. The performance of tracheal respiratory sound and SₐO₂ signal for apnea/hypopnea detection are investigated and compared when each signal is considered alone. It is shown that tracheal sound analysis had higher sensitivity than SₐO₂, while the specificity of SₐO₂ signal is higher. The combination of tracheal respiratory sounds and SₐO₂ signals is used to achieve higher sensitivity and specificity in sleep apnea detection and diagnosis.

In the system, the sound signal recordings of the entire night (after filtering the noises such as movement noises and artefacts) are available for the user (i.e., physician) to be examined by auditory and/or visual means, at the user interface of the system. To increase the processing speed of analyzing the sound signals and finding apnea/hypopnea events, the system analyses only the periods of tracheal sounds that are between a drop and rise in the SₐO₂ signal, and marks the sound segments as breath, snore and noise. The classifications are performed based on the information extracted from the normal breath sounds of the subject during the wake periods at the beginning of the recording. This self-calibration process is the only part of the method that requires input from the user. In each period, energy and duration of the classified breath segments are compared with the normal breathing periods extracted during the self-calibration stage to have a relative estimation of the total breathing volume. Duration of the classified snore segments, amplitude and the amount of drop in the SₐO₂ signal are the other features that are used to investigate the breathing quality. The weighted average of the features is calculated and thresholded to mark the apnea events.

The overall performance of the method is evaluated by comparing its AHI values (AHI_{ASAD}) with those of the PSG (AHI_{PSG}). The correlation between the outcomes of the system and PSG are found to be very high (0.9,p<0.0001). Also, the results of Bland-Altman test revealed that only 5 out of 66 subjects are outside of the 95% confidence interval, which is expected statistically. Among these 5 patients, 3 had high BMI values (43.4, 47.9 and 56.8); that is expected as the sound quality degrades when there are high amount of fat and tissue around the neck.

The AHI_{ASAD} values are used to classify the patients while the true classes are determined based on thresholding the AHI_{PSG} values as the gold standard criterion. Since, there is no standard threshold of AHI_{PSG} for classification of patients into simple snorers and OSA patients, we used the same thresholds that are most commonly used by other researchers as the threshold between the two groups. Hence, in this study we investigated grouping of the patients with the AHI_{PSG} values of 5, 10, 15 and 20 as the threshold, and determined what AHI_{ASAD} would correspond to those of PSG with the highest accuracy. The results are shown in Table III; the closer the two thresholds are, the more correlated the results of the two systems are.

For patients with mild levels of the upper airway obstruction (AHI_{PSG}≦5), the system overestimates the AHI values (as presented in FIG. 7); this justifies the low performance of the method for AHI_{PSG} threshold of 5 (Table III). When increasing the AHI_{PSG} thresholds to more than 10, the AUC becomes higher than 0.95, indicating high sensitivity and specificity. For the AHI_{PSG} thresholds of more than 20, the sensitivity and specificity of the classifiers are found to be more than 91%. This high sensitivity and specificity results is expected as the AHI values calculated by the system are highly correlated with those values calculated by PSG system. These results confirm that the calculated AHI values by the system based on only two recorded signals are good representatives of the PSG based AHI values. Thus, the ASAD system may be considered as a reliable predictor of the patient's AHI and the severity level of his/her obstruction and apnea condition. The results are found to be better than the results of the previously proposed portable monitoring devices and similar to those reported in. A detailed comparison of different methods in terms of correlation with AHI_{PSG} and classification accuracy is shown in Table IV.

While the accuracy is comparable or better than those of other current OSA monitoring systems, the main feature is that it offers relative respiratory flow estimation; this can be used for several other clinical investigations such as flow limitation in patients who may also have asthma. Furthermore, since the respiratory breath and snore sounds are recorded, they can be used to extract clinical information regarding the physiology of upper airways and breathing pattern of the patient.

Further techniques for use on breath/snore classification are described as follows. This uses three features including formants which can be added to the techniques described above to improve their accuracy.

The respiratory tracheal sounds (including snore sounds) are recorded by two Sony (ECM-77B) microphones: one placed over the suprasternal notch of the patient's neck embedded in a chamber (diameter of 6 mm) wrapped around the neck with a soft neck band (FIG. 1), and the second microphone is hung in the air about 20-30 cm from the patient's head. The sound signals are recorded simultaneously with PSG data for the entire night. The detailed analysis of their PSG data done by sleep lab technicians is used for extracting the patients' neck positions during the night.

Sound signals are amplified with a gain of 200 and band-pass filtered with the cutoff frequencies of [0.5 Hz-5 kHz] using Biopac (DA100C) amplifiers. The amplified signals are digitized at a sampling rate of 10240 Hz using N19217 data acquisition module.

It has been shown that the energy of breath sounds void of snore sounds is focused below 800 Hz, while the energy of snore sounds is up to 2000 Hz. On the other hand, snore sounds have shown to have important components in low frequencies of around 100 Hz. Therefore, the recorded sounds are band pass filtered in the frequency range 100 Hz to remove the effects of low and high-frequency noises, while including the main frequency components of both breath and snore sounds. The sound and silent segments are extracted by an automated method prior to breath and snore classification.

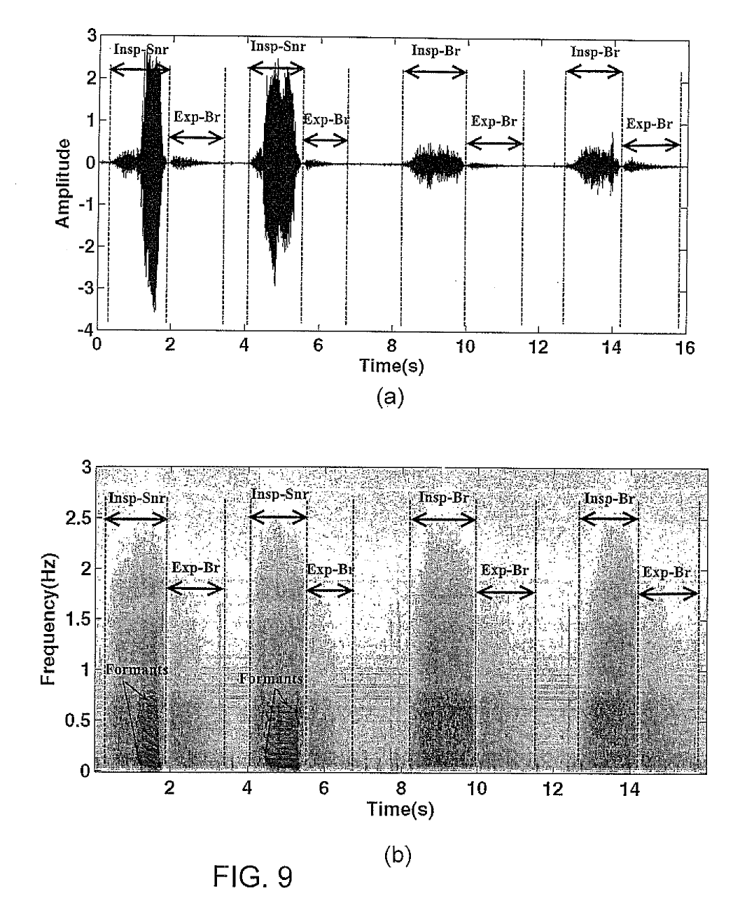

To validate the snore and breath classification method, a large number of breath and snore sound segments are first manually extracted from tracheal sounds by auditory and visual inspection of the signals in the time-frequency domain. FIG. 9 shows samples of the recorded tracheal sounds in time and time-frequency domains. Breath and snore sound segments are marked in both domains for investigating the signal's characteristics. The dark colors seen during the first and second inspirations in the time-frequency domain, represent snore sounds and its harmonic formants. These harmonics represent the resonance of the upper airways during snore sound generation. However, it should be noted that the formants are not constant among different people, and they even change from snore to snore for the same person during the night.

In order to investigate the effects of the patient's neck position, the sounds are extracted and labelled from different positions using the score sheet of PSG data. For each patient the available positions including supine (lying down, face up), prone (lying down, face down), lateral left or right are determined. Assuming symmetry between the lateral left and lateral right positions with respect to the upper airways as the source sound generation, the segments extracted from the left and right positions are merged and marked as lateral position. In total, 5909 breath and 3995 snore segments in different neck positions are extracted from all patients. The details of the number of breath and snore segments at different positions are presented in Table II.

Three features, the sound's energy in dB, zero crossing rates (ZCR and the first formant frequency (F1), are calculated from the sound segments. The number of zero crossings in each segment is calculated as:

$$ZCR = \frac{{\sum }_{k = 1}^{N - 1} \vert sign ( x ( k + 1 ) ) - sign ( x ( k ) ) \vert}{2 N} , \qquad ( 1 )$$

where N is the number of samples in each segment, sign(□) shows the sign function and |.| represents the absolute value. In each sound segment, the average of the sound signal is set to zero. Since, the number of zero crossings is proportional to the length of the signal; it is divided by N to be independent of the changes in the segment's length.

For every sound segment, linear predictive coding (LPC) is used to find the formant frequencies. In every segment, sound signal is windowed with a Hamming window of 20 ms with 50% overlap between adjacent windows and the signal in the window is estimated by an autoregressive (AR) model. Since, the first formant (F1) of the sound segments in the frequency range of below 400 Hz is found to be significantly different between breath and snore sound segments, in every window of the sound segment F1 is estimated and their median value is calculated and considered as the F1 of the sound segment.

Fisher Linear Discriminant (FLD), is used to transform the three features into a new 1D space. Principle component analysis (PCA) is another method, which is also commonly used for transforming features and extracting the best features. Calculation of the PCA base functions is a blind process, in which the class information is not considered. On the other hand, in FLD method the transform vector is estimated by maximizing the class separability. Therefore, FLD based transformation is expected to achieve better results.

In FLD method, the observations x are transformed into a new space (y=w^{T}x). In our case, x is a 3×n matrix of features extracted from the segments, and n is the number of segments. w and y are 3×1 and 1×n vectors representing the projection vector and the transformed features in the new dimension, respectively. w is estimated from the training data by maximizing the separability between classes after the projection (y).

To derive a classification threshold, we minimized the Bayesian error to estimate the optimum threshold. Assume that for a chosen threshold, the projected features that are smaller or larger than the threshold, are classified into classes ω₁ or ω₂. Then, the Bayesian error, Pₑᵣᵣ, associated with the selected threshold, k is defined as:

$${P}_{err} ( k ) = {\sum }_{{y}_{i} \geq k} p ( {y}_{i} \vert {ω}_{1} ) P ( {ω}_{1} ) + {\sum }_{{y}_{i} < k} p ( {y}_{i} \vert {ω}_{2} ) P ( {ω}_{2} ) , \qquad ( 2 )$$

where P(ω_{c}),c=1, 2 shows the probability of each class. p(yᵢ|ω_{c}),c=1, 2 is the relative probability that yᵢ actually belongs to class ω_{c},c=1, 2. Here, the probability functions are estimated by histogram functions. The optimum threshold value is determined by minimizing the error as:

$$Thr = {min}_{k} \{ {P}_{err} ( k ) \} \qquad ( 3 )$$

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. Apparatus for use in analysis of breathing pattern of a patient during sleep for detection of apnea/hypopnea events comprising:
a microphone arranged to be located on the patient for generating signals in response to breathing and snore sounds from the patient;
a finger probe Oximeter to be located on the patient's finger for recording the patient's blood SaO2 signal;
a detector module for receiving and analyzing the SaO2 signals and for receiving and analyzing the microphone signals to extract data relating to the breathing;
wherein the detector module is arranged to analyze the SaO2 signal for detecting the drops in the Oxygen level of the patient;
and wherein the detector module is arranged to analyze the microphone signals to detect breath, snore and noise sounds in response to a detected drop in the SaO2 level.

2. The apparatus according to claim 1 wherein the detector module is arranged to extract the drops in the SaO2 signal or greater than a predetermined level and to extract and analyze the breathing sounds from a limited time period starting prior to the drop of the SaO2 signal and ending at least at the end of each drop.

3. The apparatus according to claim 1 wherein the detector module is arranged to calculate from the analysis of the breathing sounds and SaO2 signal an apnea/hypopnea index.

4. The apparatus according to claim 3 wherein the index is calculated from the amplitude of SaO2 and the amount of its drop in the time period.

5. The apparatus according to claim 2 wherein the drop is at least of the order of 2%.

6. The apparatus according to claim 2 wherein the time period is at least of the order of 10 seconds before the drop.

7. The apparatus according to claim 1 wherein the detector module is arranged to extract and separate time periods into groups with snore sounds and groups without snore sounds.

8. The apparatus according to claim 1 wherein the detector module is arranged to extract and separate time periods and to divide those periods into groups with snore sounds, groups with breathing sounds and groups with noise.

9. The apparatus according to claim 8 wherein a weighted average of the groups and the SaO2 drop and amplitude are used to detect apnea/hypopnea events.

10. The apparatus according to claim 8 wherein the detector module is arranged to calculate the relative and absolute energy and duration of the sound segments to classify the sound segments into the three groups of breath, snore and noise.

11. The apparatus according to claim 10 wherein the detector module is arranged to calculate the energy, number of zero crossing rate (ZCR) and first formant of the sounds in a plurality of separate windows of data, to classify the sound segments into the groups of breath and snore.

12. The apparatus according to claim 11 wherein the detector module is arranged to use the Fisher Linear Discriminant (FLD) method to transform the three features into a new 1-dimential space and then minimize the Bayesian error to classify the sound segments into the groups of breath and snore.

13. The apparatus according to claim 1 wherein the detector module is arranged to filter extraneous sounds related to high frequency noises and/or heart sounds and movements.

14. The apparatus according to claim 1 wherein the detector module divides the microphone signals into separate windows and uses the log of the variance (LogVar) of the sound in every window of data.

15. The apparatus according to claim 1 wherein the detector module is arranged to calculate a flow estimate by the equation from the first few breaths of the patient during the wake time at a self-calibration state to estimate the relative amount of airflow for monitoring the patient's breathing pattern.

16. The apparatus according to claim 1 wherein the detector module uses an estimated breathing volume in adjacent phases to a snore phase to correctly estimate the airflow of the snore phase.

17. The apparatus according to claim 16 wherein the detector module is arranged to use the estimated airflow to detect periods of apnea and/or hypopnea.

18. The apparatus according to claim 1 wherein the detector module includes a display of the relative airflow and the detected apnea/hypopnea episodes and other statistical info for a clinician.

19. The apparatus according to claim 18 wherein the display is capable of playing the breathing and classified snoring sounds in any zoomed-in or zoomed-out data window.

20. The apparatus according to claim 1 wherein the detector module is arranged to display the extracted information about the frequency and duration of apnea/hypopnea episodes, and their association with the level of oximetry data in a separate window for the clinician.

21. The apparatus according to claim 1 wherein the microphone is wireless.

22. The apparatus according to claim 1 wherein there is provided additionally a microphone to collect lung sounds from the patient.

23. The apparatus according to claim 1 wherein there is provided a third microphone arranged to receive sounds from the patient in the vicinity of the patient so as to be sensitive to snoring and ambient noises and wherein the detector module is arranged to use adaptive filtering to extract the signals relating to the snoring and ambient noises from the signals including the breathing sounds, snoring sounds and noises.

24. The apparatus according to claim 1 wherein the microphone is arranged to collect tracheal sounds from the neck.

25. Apparatus for use in analysis of breathing pattern of a patient during sleep for detection of apnea/hypopnea events comprising:
a microphone arranged to be located on the patient for generating signals in response to breathing and snore sounds from the patient;
a detector module for receiving and analyzing the microphone signals to extract data relating to the breathing;
wherein the detector module is arranged to extract and separate time periods and to divide those periods into groups with snore sounds, groups with breathing sounds and groups with noise;
and wherein the detector module is arranged to calculate the relative and absolute energy and duration of the sound periods to classify the sound periods into the three groups of breath, snore and noise.

26. The apparatus according to claim 25 wherein the detector module is arranged to calculate the energy, number of zero crossing rate (ZCR) and first formant of the sounds in a plurality of separate windows of data, to classify the sound segments into the groups of breath and snore.

27. The apparatus according to claim 26 wherein the detector module is arranged to use the Fisher Linear Discriminant (FLD) method to transform the three features into a new 1-dimential space and then minimize the Bayesian error to classify the sound segments into the groups of breath and snore.

28. Apparatus for use in analysis of breathing pattern of a patient during sleep for detection of apnea/hypopnea events comprising:
a microphone arranged to be located on the patient for generating signals in response to breathing and snore sounds from the patient;
a detector module for receiving and analyzing the microphone signals to extract data relating to the breathing;
wherein the detector module is arranged to extract and separate time periods and to divide those periods into at least groups with snore sounds and groups with breathing sounds;
wherein the detector module uses an estimated breathing volume in adjacent phases to a snore phase to correctly estimate the airflow of the snore phase.

28. The apparatus according to claim 27 wherein the detector module is arranged to use the estimated airflow to detect periods of apnea and/or hypopnea.
